(19) United States
(12) Patent Application Publication
(10) Pub. No.: US 2017/0027615 A1
(43) Pub. Date: Feb. 2, 2017

(54) SPINAL IMPLANT SYSTEM AND METHOD

(71) Applicant: Warsaw Orthopedic, Inc., Warsaw, IN (US)

(72) Inventor: William Alan Rezach, Atoka, TN (US)

(21) Appl. No.: 14/814,787

(22) Filed: Jul. 31, 2015

(51) Int. Cl. A61B 17/70

(52) U.S. Cl. CPC A61B 17/7043; A61B 17/7032; A61B 17/7037

(57) ABSTRACT

A spinal construct comprises a fastener attached with sacral and/or pelvic tissue of a body. A first connector includes a mating element engageable with the fastener and disposable in a fixed orientation therewith. A second connector is connected with a spinal implant and includes a mating element engageable with the first connector. The second connector is selectively adjustable relative to the first connector and disposable in a fixed orientation therewith. Systems and methods are disclosed.

### TECHNICAL FIELD

The present disclosure generally relates to surgical implants for the treatment of spinal disorders, and more particularly to a surgical system and method for treatment of a spine disorder.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, correction, discectomy, laminectomy and implantable prosthetics. As part of these surgical treatments, spinal constructs, such as, for example, bone fasteners, spinal rods, connectors and plates can be used to provide stability to a treated region. During surgical treatment, one or more rods may be attached via fasteners and connectors to the exterior of two or more vertebral members to provide stability to a treated region. Fasteners may also be attached to iliac bone. This disclosure describes an improvement over these prior technologies.

### SUMMARY

In one embodiment, a spinal construct is provided. The spinal construct comprises a fastener attached with sacral and/or pelvic tissue of a body. A first connector includes a mating element engageable with the fastener and disposable in a fixed orientation therewith. A second connector is connected with a spinal implant and includes a mating element engageable with the first connector. The second connector is selectively adjustable relative to the first connector and disposable in a fixed orientation therewith. In some embodiments, systems and methods are provided.

### BRIEF DESCRIPTION OF THE DRAWINGS

The present disclosure will become more readily apparent from the specific description accompanied by the following drawings, in which:

FIG. 1 is a perspective view of components of one embodiment of a surgical system in accordance with the principles of the present disclosure;

FIG. 2 is a perspective view of components of the surgical system shown in FIG. 1;

FIG. 3 is a perspective view of components of one embodiment of a surgical system in accordance with the principles of the present disclosure;

FIG. 4 is a perspective view of components of one embodiment of a surgical system in accordance with the principles of the present disclosure;

FIG. 5 is a plan view of components of one embodiment of a surgical system in accordance with the principles of the present disclosure;

FIG. 6 is a perspective view of components of one embodiment of a surgical system in accordance with the principles of the present disclosure;

FIG. 7 is a break away perspective view of components of one embodiment of a surgical system in accordance with the principles of the present disclosure;

FIG. 8 is a break away perspective view of components of one embodiment of a surgical system in accordance with the principles of the present disclosure;

FIG. 9 is a perspective view of the components shown in FIG. 8;

FIG. 10 is a side view of the components shown in FIG. 9;

FIG. 11 is a side view of the components shown in FIG. 9;

FIG. 12 is a perspective view of components of one embodiment of a surgical system in accordance with the principles of the present disclosure;

FIG. 13 is a perspective view of the components shown in FIG. 12; and

FIG. 14 is a perspective view of components of one embodiment of a surgical system in accordance with the principles of the present disclosure disposed with vertebrae.

### DETAILED DESCRIPTION

The exemplary embodiments of the surgical system and related methods of use disclosed are discussed in terms of medical devices for the treatment of musculoskeletal disorders and more particularly, in terms of a surgical system and method for treatment of a spine disorder. In some embodiments, the systems and methods of the present disclosure are employed with a spinal joint fusion, for example, with a cervical, thoracic, lumbar and/or sacral region of a spine.

In some embodiments, the present disclosure provides a surgical system comprising a spinal construct that includes an iliac screw connector. In some embodiments, the connector has slidable medial/lateral adjustment and angulation. In some embodiments, the connector has a pop-on design. In some embodiments, the present disclosure provides a surgical system comprising a spinal construct that is employed with a method for treating a spine including low profile iliac fixation.

In some embodiments, the present disclosure provides a surgical system comprising a spinal construct that includes a low profile multi-axial receiver configuration for iliac screws. In some embodiments, the spinal construct has a receiver with a built in plate or rod for connection to a primary rod. In some embodiments, the spinal construct includes medial/lateral adjustment away from an iliac screw on a primary rod attachment. In some embodiments, the spinal construct has a receiver with a minimal profile under a primary rod. In some embodiments, the spinal construct has the ability to rotate in a coronal plane of a body relative to a primary rod.

In some embodiments, the present disclosure provides a surgical system comprising a spinal construct that includes an iliac screw, an iliac connector/receiver and a sacral, for example, S1 vertebral level multi-axial screw. In some embodiments, the present disclosure provides a surgical system comprising a spinal construct that is employed with a method for treating a spine including placing an iliac screw with tissue. In some embodiments, the iliac screw includes a head without an implant receiver. In some embodiments, the method includes the step of placing a spinal rod with one or more fasteners connected with tissue. In some embodiments, the method includes the step of sliding a rod connector onto an end of the rod. In some embodiments, a surgical instrument may be used to facilitate disposing the rod connector with the rod. In some embodiments, the method includes the step of selecting a connector/receiver and sliding the connector/receiver onto a head of an iliac screw and the rod connector. In some embodiments, a rod connecting loading instrument guides components of the spinal construct for assembly.

In some embodiments, the method includes the step of securing the components of the spinal construct with set screws, for example, a set screw engages the connector/receiver and the head of the iliac screw and a set screw engages the connector/receiver and the rod connector. In some embodiments, the spinal construct has a minimal profile above the spinal rod and/or a low profile under the spinal rod. In some embodiments, the method includes the step of adjusting the components of the spinal construct in a medial/lateral orientation, for example, translating the connector/receiver relative to the rod connector for medial/lateral adjustment over the spinal rod and adjustment is avoided near the iliac crest.

In some embodiments, the rod connector may include a washer that has flats and/or indicia to display selective orientation of the rod connector. In some embodiments, the washer includes flats and/or indicia to display mating engagement of the rod connector with the rod. In some embodiments, the rod connector includes a body and a washer. In some embodiments, the washer can be pre-assembled with the body. In some embodiments, the washer includes a stake hole and is staked with the body. In some embodiments, the washer is welded with the body. In some embodiments, the washer is movable and/or translatable vertically in an up and down direction.

In one embodiment, the present disclosure provides a surgical system including a spinal construct configured to allow for coronal adjustability, such as, for example, in a sacro-pelvic region. In one embodiment, the spinal construct includes a connector that allows for coronal adjustability. In one embodiment, a spinal construct is configured to allow for coronal adjustability to facilitate assembly of the construct including a rod extending from a lumbar spine to an iliac and/or sacro-iliac screw.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, micro discectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, muscle, ligaments, tendons, cartilage and/or bone unless specifically referred to otherwise.

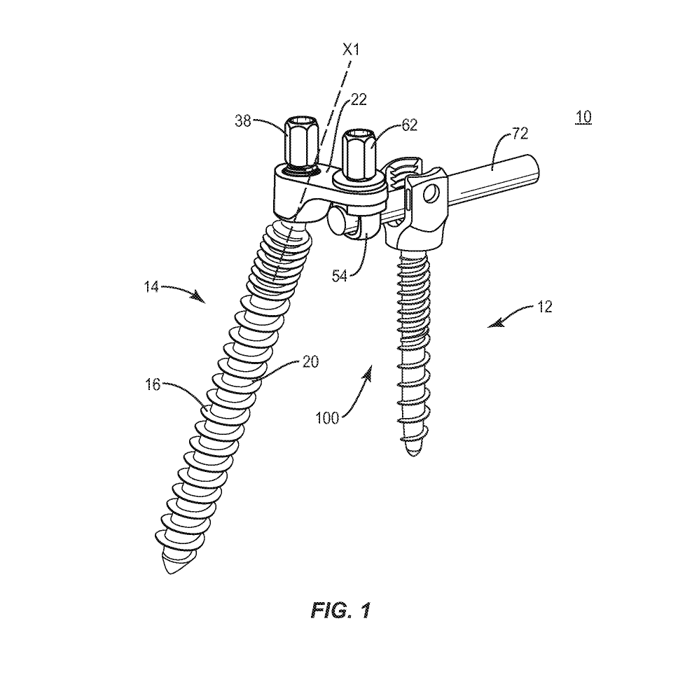

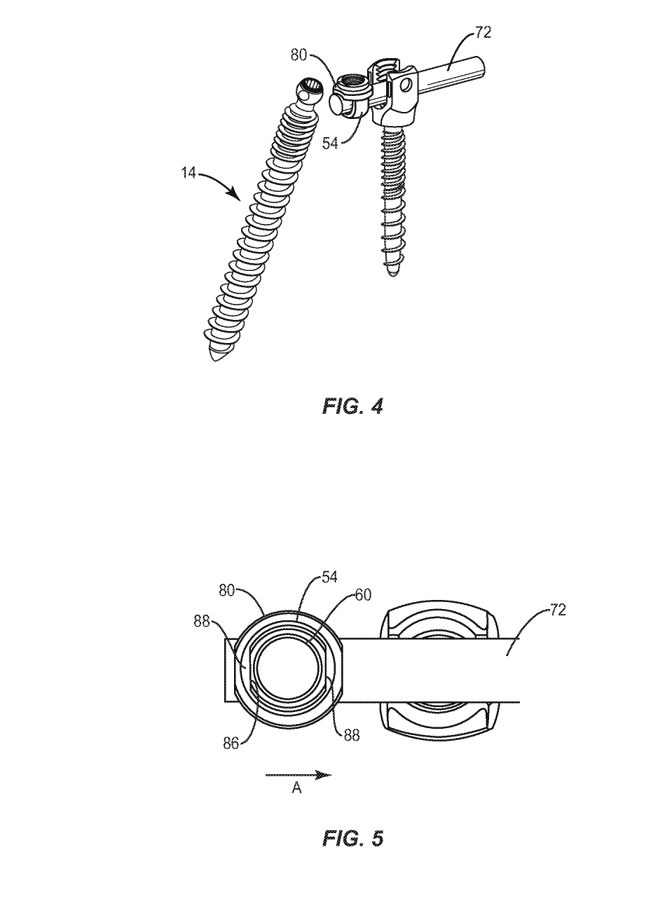

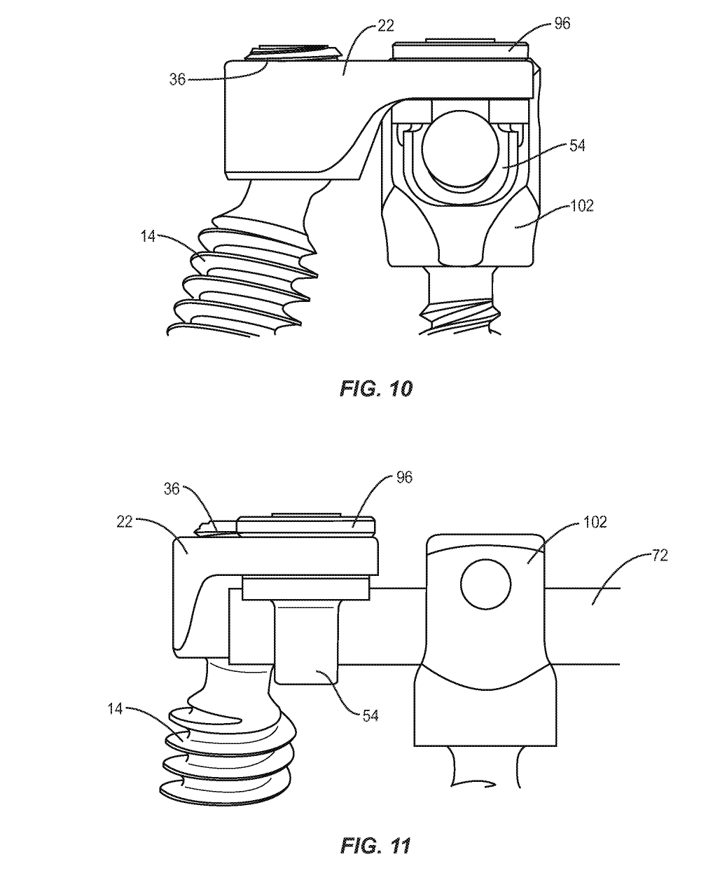

The following discussion includes a description of a surgical system and related methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-13, there are illustrated components of a surgical system, such as, for example, a spinal implant system 10.

The components of spinal implant system 10 can be fabricated from biologically acceptable materials suitable for medical applications, including metals, synthetic polymers, ceramics and bone material and/or their composites. For example, the components of spinal implant system 10, individually or collectively, can be fabricated from materials such as stainless steel alloys, commercially pure titanium, titanium alloys, Grade 5 titanium, super-elastic titanium alloys, cobalt-chrome alloys, super-elastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaS04 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TCP), HA-TCP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-β), osteoblast cells, growth and differentiation factor (GDF), insulin-like growth factor 1, platelet-derived growth factor, fibroblast growth factor, or any combination thereof.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein. In one embodiment, a spinal implant, as described herein, may be formed substantially of biocompatible polymer, such as PEEK, and selectively coated with a biocompatible metal, such as titanium, or a bone-growth promoting material, such as HA. In some embodiments, titanium may be plasma sprayed onto surfaces of the spinal implant to modify a radiographic signature of the spinal implant and/or improve bony ongrowth to the spinal implant by application of a porous or semi-porous coating of titanium.

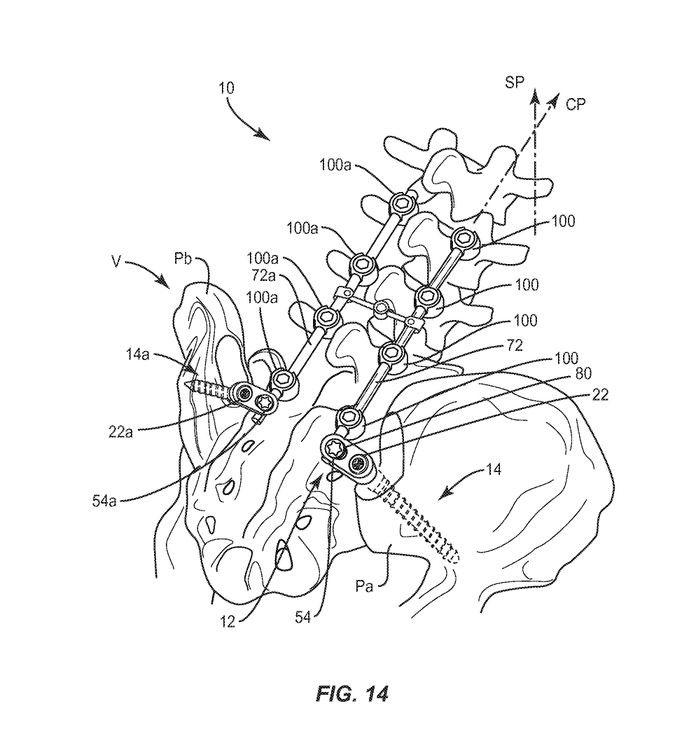

Spinal implant system 10 may be employed, for example, with minimally invasive procedures, including percutaneous techniques, mini-open surgical techniques and/or open surgical techniques to deliver and introduce instrumentation and/or implants, such as, for example, a spinal construct, at a surgical site within a subject body of a patient, which includes, for example, a spine having vertebrae V, as shown, for example, in FIG. 14. In some embodiments, the spinal constructs can include one or more bone fasteners, spinal rods, connectors, vertebral replacement devices, interbody devices and/or plates.

Spinal implant system 10 includes a spinal construct 12 comprising a fastener, such as, for example, an iliac bone screw 14 configured for attachment with sacral and/or pelvic tissue of a body. Bone screw 14 includes a shaft 16 and a head 18. Bone screw 14 defines an axis X1. Shaft 16 is configured to penetrate tissue. Shaft 16 has a cylindrical cross section and includes an outer surface 20 having an external thread form. In some embodiments, the thread form of surface 20 may include a single thread turn or a plurality of discrete threads. In some embodiments, the thread form of surface 20 may be self-tapping or intermittent, or may have more than one crest winding about shaft 16. In some embodiments, other engaging structures may be disposed on shaft 16, such as, for example, a nail configuration, barbs, expanding elements, raised elements and/or spikes to facilitate engagement of shaft 16 with tissue, such as, for example, vertebrae. In some embodiments, all or only a portion of shaft 16 may have alternate cross section configurations, such as, for example, oval, oblong, triangular, square, polygonal, irregular, uniform, non-uniform, offset, staggered, undulating, arcuate, variable and/or tapered. In some embodiments, all or only a portion of surface 20 may have alternate surface configurations to enhance fixation with tissue, such as, for example, rough, arcuate, undulating, mesh, porous, semi-porous, dimpled and/or textured. In some embodiments, all or only a portion of shaft 16 may be cannulated.

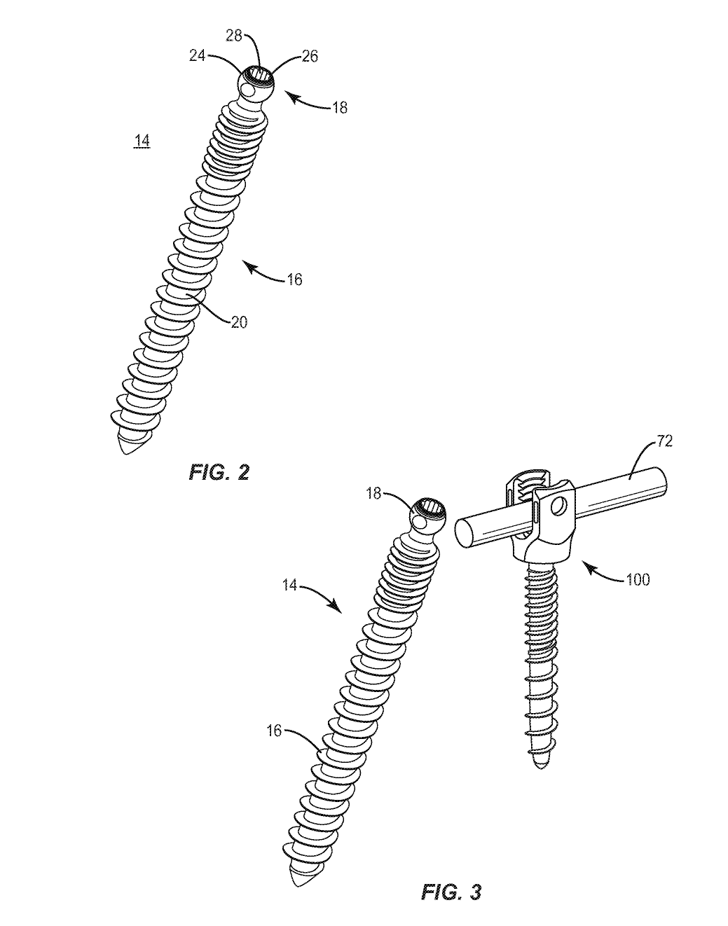

Head 18 is engageable with a connector, such as, for example, a multi-axial connector 22, as described herein. Head 18 includes a substantially spherical configuration configured for moveable disposal with connector 22. Head 18 includes a surface 24 that defines a plurality of ridges 26, as shown in FIG. 2, to improve purchase of head 18 with connector 22 for disposal of bone screw 14 in a selected orientation with an relative to connector 22. Head 18 includes a socket 28 having a hexalobe geometry configured for disposal of a similarly shaped bit of a tool, such as, for example, a driver (not shown) to engage the driver with head 18 to rotate shaft 16. In some embodiments, socket 28 has a cruciform, phillips, square, hexagonal, polygonal, star cross sectional configuration for disposal of a correspondingly shaped portion of the driver.

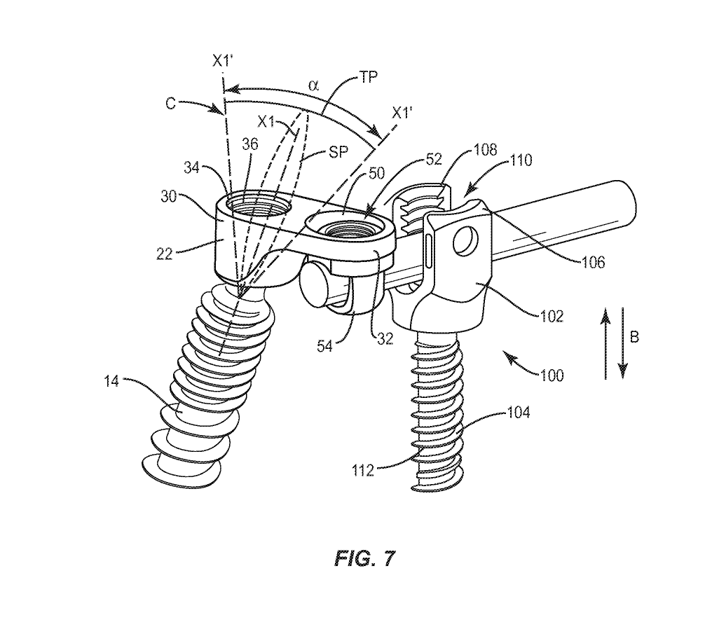

Connector 22 extends between an end 30 and an end 32, as shown in FIG. 7. Connector 22 includes a surface 34 that defines a mating element, such as, for example, an opening 36 disposed at end 30. Opening 36 is configured for disposal of head 18. In some embodiments, opening 36 includes and surface 34 defines a socket configured for disposal of head 18 in a mufti-axial movement configuration. In some embodiments, surface 34 defines a cavity for disposal of head 18 to comprise a spheroidal joint to facilitate multi-axial movement of bone screw 14 relative to connector 22, as described herein. A portion of surface 34 is threaded for engagement with a lock, such as, for example, a breakoff set screw 38 configured to fix bone screw 14 with connector 22 in a selected orientation.

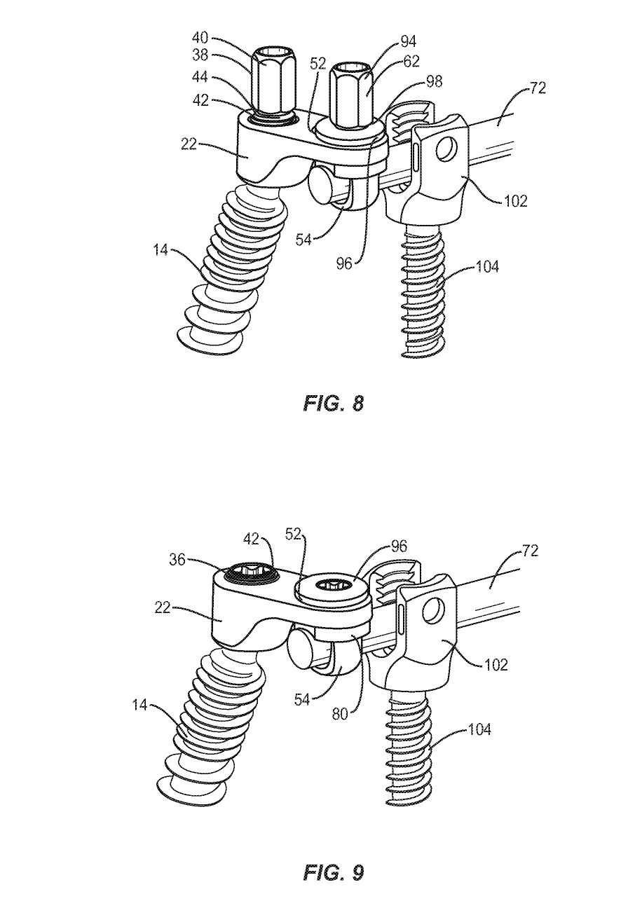

Setscrew 38 includes a portion 40 and a portion 42, as shown in FIG. 8. Portions 40, 42 are connected at a reduced diameter portion 44 that is frangibly connected to portion 42. In some embodiments, portion 42 is configured for engagement with opening 36. In some embodiments, portions 40, 42 are fabricated from a fracturing and/or frangible material such that manipulation of portion 40 relative to portion 42 can fracture and separate portion 40 from portion 42 at a predetermined force and/or torque limit, as described herein. In some embodiments, as force and/or torque is applied to portion 40 and resistance increases, for example, due to fixation of portion 42 with opening 36, as described herein, the predetermined torque and force limit is approached.

In some embodiments, all or only a portion of surface 34 may have alternate cross section configurations, such as, for example, oval, oblong, triangular, square, polygonal, irregular, uniform, non-uniform, offset, staggered, undulating, arcuate, variable and/or tapered. In some embodiments, surface 34 may have alternate surface configurations, such as, for example, rough, arcuate, undulating, mesh, porous, semi-porous, dimpled and/or textured.

In some embodiments, head 18 is configured for disposal with opening 36 between a configuration such that bone screw 14 and/or connector 22 is relatively rotatable and/or pivotable from an initial orientation, for example, connector 22 being disposed at an orientation relative to bone screw 14, for example, as shown by axis X1, and rotatable and/or pivotable to a selected orientation relative to bone screw 14, for example, as shown by one or a plurality of axes X1′ relative to axis X1 in FIG. 7. In some embodiments, bone screw 14 is rotatable and/or pivotable between the orientations relative to connector 22 in a multi-axial configuration. In some embodiments, bone screw 14 is rotatable and/or pivotable between the orientations relative to connector 22 in a uni-axial configuration. In some embodiments, bone screw 14 is movably fixed relative to connector 22.

In some embodiments, bone screw 14 and/or connector 22 is relatively rotatable and/or pivotable, as described herein, to a selected angle through and within angular range a in a plurality of planes that lie in a cone configuration C. In some embodiments, bone screw 14 and/or connector 22 is relatively rotatable and/or pivotable, as described herein, to a selected angle within angular range a in sagittal plane SP, corresponding to a particular plane that lies in cone C. In some embodiments, bone screw 14 and/or connector 22 is relatively rotatable and/or pivotable, as described herein, to a selected angle within angular range a in transverse plane TP, corresponding to a particular plane that lies in cone C. In some embodiments, bone screw 14 and/or connector 22 is relatively rotatable and/or pivotable, as described herein, to a selected angle within angular range a in coronal plane CP, corresponding to a particular plane that lies in cone C. In some embodiments, bone screw 14 is fixed with connector 22 in one or a plurality of selected orientations, as described herein.

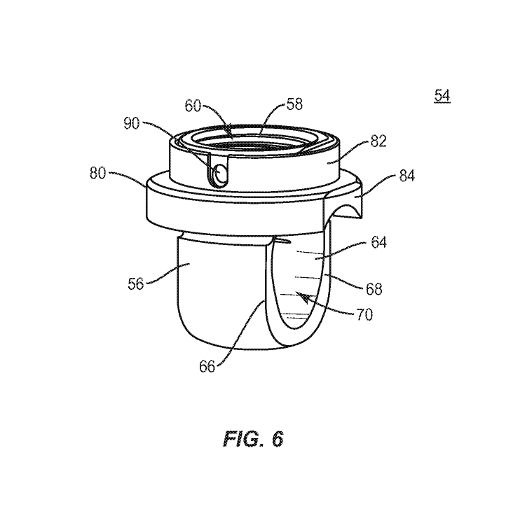

Connector 22 includes a surface 50 that defines a mating element, such as, for example, a slot 52 disposed at end 32. Slot 52 is configured for disposal of a connector, such as, for example, a rod connector 54, as shown in FIG. 6. Slot 52 is configured to facilitate selective movement of connector 54 within slot 52 relative to connector 22, as described herein. In some embodiments, all or only a portion of slot 52 may have alternate cross section configurations, such as, for example, oval, oblong, triangular, square, polygonal, irregular, uniform, non-uniform, offset, staggered, undulating, arcuate, variable and/or tapered. In some embodiments, surface 50 may have alternate surface configurations, such as, for example, rough, arcuate, undulating, mesh, porous, semi-porous, dimpled and/or textured. In some embodiments, spinal implant system 10 includes a spinal implant kit having a plurality of alternate connectors 22 that are compatible and/or interchangeable with bone screw 14 and/or rod connector 54. In some embodiments, spinal implant system 10 includes a spinal implant kit having a plurality of alternate rod connectors 54 that are compatible and/or interchangeable with bone screw 14 and/or connector 22.

Connector 54 includes a body 56. Body 56 includes a surface 58 that defines an opening 60. Opening 60 is configured for engagement with a lock, such as, for example, a breakoff set screw 62 (FIG. 1). A portion of surface 58 is threaded to facilitate engagement with set screw 62. Body 56 includes a surface 64 that defines spaced arms 66, 68. Arms 66, 68 define a U-shaped cavity 70 therebetween configured for disposal of a spinal implant, such as, for example, a spinal rod 72. In some embodiments, cavity 70 may have various cross section configurations, such as, for example, oval, oblong, triangular, rectangular, square, polygonal, irregular, uniform, non-uniform, variable, tubular and/or tapered. In some embodiments, arm 66, arm 68 and/or cavity 70 may be disposed at alternate orientations, such as, for example, transverse, perpendicular and/or other angular orientations such as acute or obtuse, co-axial and/or may be offset or staggered. In some embodiments, cavity 70 is closed to define a closed cavity 70.

Connector 54 includes a mating element, such as, for example, a washer 80. In some embodiments, washer 80 includes a portion 82 and a portion 84 connected in a stepped configuration. Washer 80 includes a surface 86 that includes visual indicia, such as, for example, planar surfaces 88 configured to indicate alignment of cavity 70 with spinal rod 72, as described herein. Washer 80 is configured for translation relative to body 56 to facilitate connection with spinal rod 72. In some embodiments, planar surfaces 88 indicate spinal rod 72 insertion orientation. In some embodiments, the indicia may include one or more ribs, spikes, bosses, or protrusions that interact with spinal rod 72 to achieve a tactile indication. In some embodiments, body 56 includes a cavity, such as, for example, recess 90 configured facilitate assembly of washer 80 with body 56, such as, for example, washer 80 is staked to deform washer 80 into engagement with body 56 and mating engagement with recess 90. In some embodiments, washer 80 is welded to body 56.

Connector 54 is selectively adjustable relative to connector 22 and is configured for disposal in a fixed orientation relative to connector 22. In some embodiments, connector 54 is axially translatable relative to connector 22 within slot 52. In some embodiments, connector 54 is configured for translation within slot 52 to facilitate selective positioning of connector 54 relative to connector 22 in a medial lateral orientation within coronal plane CR In some embodiments, translation of connector 54 is positioned over spinal rod 72 to avoid and/or prevent movement over the iliac crest. Connector 54 is configured for fixation with connector 22 with set screw 62, as described herein.

Setscrew 62 includes a portion 94 and a portion 96. Portions 94, 96 are connected at a reduced diameter portion 98 that is frangibly connected to portion 96. In some embodiments, portion 94 is configured for engagement with opening 60. In some embodiments, portions 94, 96 are fabricated from a fracturing and/or frangible material such that manipulation of portion 94 relative to portion 96 can fracture and separate portion 94 from portion 96 at a predetermined force and/or torque limit, as described herein. In some embodiments, as force and/or torque is applied to portion 94 and resistance increases, for example, due to fixation of portion 96 with opening 60, as described herein, the predetermined torque and force limit is approached.

In some embodiments, spinal implant system 10 includes a bone screw, such as, for example, a multi-axial screw 100. Screw 100 includes a receiver 102 configured for disposal of spinal rod 72 and a shaft 104 configured to penetrate tissue. Receiver 102 includes a pair of spaced apart arms 106, 108. Arms 106, 108 define a U-shaped implant cavity 110 therebetween configured for disposal of spinal rod 72, as described herein. In some embodiments, cavity 110 may have various cross section configurations, such as, for example, oval, oblong, triangular, rectangular, square, polygonal, irregular, uniform, non-uniform, variable, tubular and/or tapered. In some embodiments, arm 106, arm 108 and/or cavity 110 may be disposed at alternate orientations, relative In each other, such as, for example, transverse, perpendicular and/or other angular orientations such as acute or obtuse, co-axial and/or may be offset or staggered. In some embodiments, receiver 102 may be dosed to define a dosed cavity 110. In some embodiments, a coupling member, such as, for example, a set screw may engage receiver 102 to fix and/or lock spinal rod 72 with receiver 102. In some embodiments, receiver 102 includes a low profile between shaft 104 and spinal rod 72.

Shaft 104 is configured with a cylindrical cross section and includes an outer surface 112 having an external thread form. In some embodiments, the thread form on surface 112 may include a single thread turn or a plurality of discrete threads. hi some embodiments, the thread form on surface 112 may be self-tapping or intermittent, or may have more than one crest winding about shaft 104. In some embodiments, other engaging structures may be disposed on shaft 104, such as, for example, a nail configuration, barbs, expanding elements, raised elements and/or spikes to facilitate engagement of shaft 104 with tissue, such as, for example, vertebrae. In some embodiments, all or only a portion of shaft 104 may have alternate cross section configurations, such as, for example, oval, oblong, triangular, square, polygonal, irregular, uniform, non-uniform, offset, staggered, undulating, arcuate, variable and/or tapered. In some embodiments, all or only a portion of surface 112 may have alternate surface configurations to enhance fixation with tissue such as, for example, rough, arcuate, undulating, mesh, porous, semi-porous, dimpled and/or textured. In some embodiments, all or only a portion of shaft 104 may be cannulated.

In assembly, operation and use, spinal implant system 10, similar to the systems and methods described herein, is employed with a surgical procedure, as described herein, for treatment of a condition or injury of an affected section of the spine including vertebrae V, as shown in FIG. 14.

In use, to treat the affected section of the spine, a medical practitioner obtains access to a surgical site including vertebrae V and/or a sacro-pelvic region of a body in any appropriate manner, such as through incision and retraction of tissues. Spinal implant system 10 may be used in any existing surgical method or technique including open surgery, mini-open surgery, minimally invasive surgery and percutaneous surgical implantation, whereby portions of vertebrae V and/or a sacro-pelvic region of a body are accessed through one or more micro-incisions, or sleeves that provide a protected passageway to the area. Once access to the surgical site is obtained, the particular surgical procedure is performed for treating the spinal disorder. Spinal implant system 10 is employed to augment the surgical treatment. One or more of the components of spinal implant system 10 can be delivered or implanted as a pre-assembled device or can be assembled in situ.

Pilot holes are made in vertebrae V and screws 100 are disposed along vertebrae V. Pilot holes are made in a sacro-pelvic region, such as, for example, ilium Pa and ilium Pb. Bone screw 14 is fastened with the tissue of ilium Pa. Spinal rod 72 is disposed with screws 100 and attached along a lateral side of vertebrae V. Rod connector 54 is translated onto spinal rod 72, in a direction shown by arrow A in FIG. 5. In some embodiments, an instrument (not shown) is connected with rod connector 54 to facilitate connection with spinal rod 72. Washer 80 is oriented such that planar surfaces 88 indicate correct positioning of spinal rod 72 with rod connector 54, as shown in FIG. 5, Connector 22 is selected and translated onto head 18 of bone screw 14 such that head 18 is disposed with opening 36, as described herein. In some embodiments, a rod connector 54 can be selected from a kit and/or plurality of compatible and/or interchangeable components of spinal implant system 10, as described herein.

Connector 22 is rotatable and/or pivotable to a plurality of axes relative to axis X1, as described herein. In some embodiments, connector 22 is rotatable and/or pivotable to a selected angle through and within angular range a relative to axis X1, as described herein and shown, for example, in FIG. 7. Rotation of connector 22 relative to bone screw 14 facilitates engagement and disposal of rod connector 54 with connector 22.

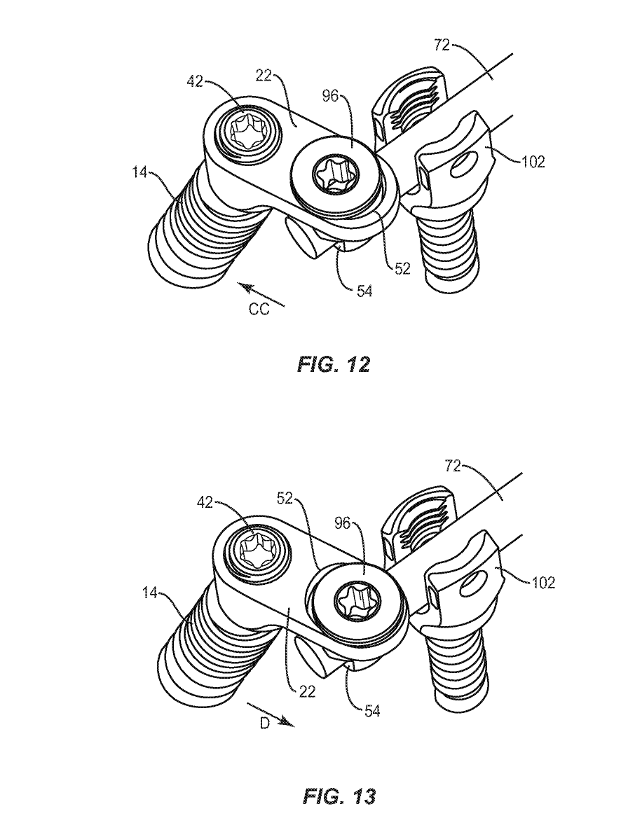

Rod connector 54 is engaged with slot 52. Washer 80 and/or body 56 are manipulated to facilitate connection of connector 22 with spinal rod 72. Washer 80 is configured for translation, in a direction shown by arrows B in FIG. 7, relative to connector 22 to facilitate positioning of spinal rod 72 with connector 22. Rod connector 54 translates within slot 52, in a direction shown by arrow CC in FIG. 12 and arrow D in FIG. 13, to facilitate selective medial-lateral adjustment over spinal rod 72 and a distance from bone screw 14. Setscrew 38 is engaged with opening 36 to fix bone screw 14 with connector 22. Setscrew 62 is engaged with rod connector 54 to fix rod connector 54 and spinal rod 72 with connector 22. In some embodiments, setscrew 62 provisionally fixes rod connector 54 with connector 22 to allow for adjustment and is further engaged for final fixation.

In some embodiments, spinal implant system 10 includes spinal rod 72a disposed along and attached with screws 100a to a contra lateral side of vertebrae V, similar to spinal rod 72 and screws 100 disposed with the lateral side of vertebrae V and described above. Spinal rod 72a is connected with a spinal construct 12a via rod connector 54a and connector 22a and which includes bone screw 14a attached ilium Pb, similar to spinal construct 12.

Upon completion of the procedure, the surgical instruments, assemblies and non-implanted components of spinal implant system 10 are removed and the incision is dosed. Spinal implant system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal implant system 10. In some embodiments, spinal implant system 10 may include one or a plurality of plates, connectors, vertebral replacement devices, interbody devices and/or bone fasteners for use with a single vertebral level or a plurality of vertebral levels.

In some embodiments, spinal implant system 10 includes one or more fasteners for attaching a spinal construct with tissue, as described herein. In some embodiments, the fasteners may be engaged with tissue in various orientations, such as, for example, series, parallel, offset, staggered and/or alternate vertebral levels. In some embodiments, one or more of the fasteners may comprise multi-axial screws, sagittal angulation screws, pedicle screws, mono-axial screws, uni-planar screws, facet screws, fixed screws, tissue penetrating screws, conventional screws, expanding screws, wedges, anchors, buttons, dips, snaps, friction fittings, compressive fittings, expanding rivets, staples, nails, adhesives, posts, fixation plates and/or posts.

In some embodiments, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the components and/or surfaces of spinal implant system 10 with vertebrae. In some embodiments, the agent may include one or a plurality of therapeutic agents and/or pharmacological agents for release, including sustained release, to treat, for example, pain, inflammation and degeneration.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal construct comprising:
a fastener attached with sacral and/or pelvic tissue of a body;
a first connector including a mating element engageable with the fastener and disposable in a fixed orientation therewith;
a spinal implant; and
a second connector connected with the spinal implant and including a mating element engageable with the first connector, the second connector being selectively adjustable relative to the first connector and disposable in a fixed orientation therewith.

2. A spinal construct as recited in claim 1, wherein the second connector is axially translatable relative to the first connector.

3. A spinal construct as recited in claim 1, wherein the second connector is selectively movable relative to the first connector in a coronal plane.

4. A spinal construct as recited in claim 1, wherein the first connector defines a slot and the second connector is selectively movable within the slot.

5. A spinal construct as recited in claim 1, wherein the first connector is rotatable relative to the second connector in a coronal plane.

6. A spinal construct as recited in claim 1, wherein the second connector is selectively axially translatable relative to the first connector in a medial/lateral orientation within a coronal plane.

7. A spinal construct as recited in claim 1, wherein the fastener defines a longitudinal axis and the first connector is rotatable to a plurality of axes relative to the longitudinal axis.

8. A spinal construct as recited in claim 1, further comprising a set screw engageable with the first connector and the fastener to fix the first connector relative to the fastener.

9. A spinal construct as recited in claim 1, wherein the mating element of the second connector comprises a stepped washer.

10. A spinal construct as recited in claim 1, wherein the second connector includes spaced arms that define a U-shaped implant cavity.

11. A spinal construct as recited in claim 10, wherein the second connector includes visual indicia of alignment of the implant cavity with the spinal implant.

12. A spinal construct as recited in claim 1, wherein the second connector includes a washer mounted with a body configured for disposal of the spinal implant.

13. A spinal construct as recited in claim 1, further comprising a set screw engageable with the second connector and the first connector to fix the first connector relative to the second connector.

14. A spinal construct as recited in claim 1, wherein the spinal implant includes a spinal rod connected with vertebral tissue via at least one multi-axial screw.

15. A spinal construct comprising:
an iliac bone screw;
a multi-axial connector engageable with the screw and disposable in a fixed orientation therewith;
a spinal rod connected with at least one multi-axial bone screw; and
a rod connector engageable with the multi-axial connector and defining an implant cavity configured for disposal of the spinal rod, the rod connector being selectively adjustable relative to the multi-axial connector and disposable in a fixed orientation therewith.

16. A spinal construct as recited in claim 15, wherein the multi-axial connector defines a slot and the rod connector is selectively movable within the slot.

17. A spinal construct as recited in claim 15, wherein the rod connector is selectively axially translatable relative to the multi-axial connector in a medial/lateral orientation within a coronal plane.

18. A spinal implant system comprising:
a fastener attached with sacral and/or pelvic tissue of a body;
a plurality of alternate first connectors, each of the first connectors including a mating element engageable with the fastener such that the fastener is compatible with the plurality of first connectors and disposable in a fixed orientation therewith;
a spinal implant; and
a second connector connected with the spinal implant and including a mating element engageable with the first connectors such that the second connector is compatible with the plurality of first connectors, the second connector being selectively adjustable relative to the first connectors and disposable in a fixed orientation therewith.

19. A spinal construct as recited in claim 18, wherein the spinal implant includes a spinal rod connected with vertebral tissue via at least one multi-axial screw.

20. A spinal construct as recited in claim 18, further comprising a guide instrument for assembling the connectors.
